(19) United States
(12) Patent Application Publication
(10) Pub. No.: US 2025/0032792 A1
(43) Pub. Date: Jan. 30, 2025

(54) SERIALISED OR PARALLELISED BI-CHANNEL HIGH-FREQUENCY ELECTROTHERAPY DEVICE

(71) Applicant: WINBACK GROUP, Biarritz (FR)

(72) Inventors: Christophe BUÉE, Saint Jean De Luz (FR); Gilles JOVÈNE, St Laurent Du Var (FR); Sebastien BROUCHET, Beaulieu Sur Mer (FR); Guillaume JEAMMOT, Nice (FR)

(73) Assignee: WINBACK GROUP, Biarritz (FR)

(21) Appl. No.: 18/834,789

(22) PCT Filed: Feb. 2, 2023
(86) PCT No.: PCT/EP2023/052601

§ 371 Date: Jul. 31, 2024

(30) Foreign Application Priority Data
Feb. 3, 2022 (FR) FR2200974
Feb. 3, 2022 (FR) FR2200975

(51) Int. Cl. A61N 1/36; A61N 1/04; H02M 7/44

(52) U.S. Cl. CPC A61N 1/36034; A61N 1/0404; H02M 7/44

(57) ABSTRACT

An electrotherapy device comprising: N electrodes, N being equal to 2 or 3, two sinusoidal-voltage generators, characterized in that the two voltage generators are isolated by a transformer and the device comprises a control unit configured so that the device takes in alternation: a first so-called series configuration wherein the N equal to 2 electrodes being active electrodes and the two voltage generators are put in series, the device does not comprise a return electrode, a second so-called parallel configuration wherein N equal to 3, two electrodes of which are active electrodes each respectively connected to a voltage generator and one electrode is a return electrode to form the ground and is connected to each voltage generator, the two voltage generators are put in parallel.

### TECHNICAL FIELD

The present invention relates to an electronic apparatus for therapeutic or cosmetic use. The invention will more particularly find its application in the so-called electrotherapy field, in particular for treatment by diathermy and/or conductivity.

### PRIOR ART

Electrotherapy is a non-hazardous non-invasive technique that consists in using electricity for a therapeutic purpose. This technique is recognised for easing pain, strengthening muscle fibres or accelerating healing of biological tissues. Three major families of frequencies exist, such as low frequencies (1 Hz-150 Hz) for superficial neurostimulating action, medium frequencies (1 kHz-1 kHz) for deep neurostimulating action and finally high frequencies (100 kHz-1.2 MHz) for superficial or deep selective diathermal action and acceleration of healing. Low frequencies (LF) and medium frequencies (MF) are generally designated as electrostimulation and high frequencies (HF) are designated as radio frequency.

These various types of electrotherapy current circulate between two plates or conductive elements that fulfil the role of electrodes in contact with the skin. Various types of current can be used by the practitioner.

Heat is a therapeutic modality that has been used for many years in kinesitherapy and is divided into two categories: superficial heating agents and deep heating agents. The modalities of deep thermotherapy include long-wave and short-wave therapeutic or cosmetic diathermy, ultrasound and contact radio frequency, the latter also being referred to as high-frequency current, which is between 100 kHz and 1.2 MHz. This type of deep thermotherapy is called diathermy. Diathermy creates heating in the cell tissues of the injured parts of the body between two electrodes in contact with the living tissue, so that a circulation of electric current occurs in the body between these two electrodes.

By definition, current-conduction diathermy equipment includes an active electrode and a return electrode, as disclosed for example in the patent ES 287 964.

Because of the electrical impedance of the tissue itself, the electric current circulates through the tissue and causes a rise in temperature of the tissue by Joule effect. This heating is appreciable and is related to the increase in intensity of the current.

The conductivity of the tissue changes as a function of the high-frequency current. The higher the voltage, the more conductive is the tissue and the more accelerated the healing thereof.

When there are a plurality of electrodes, they are connected to a single high-frequency voltage generator, which favours the diathermy effects and the healing of the tissues at the same time in the treatment. These two effects are therefore combined and cannot be dissociated.

The document U.S. Pat. No. 5,776,173 A1 is known, which proposes an interferential stimulation apparatus for electrotherapy. The apparatus comprises two oscillators each generating an output signal having a frequency, the output signal passes through several components before passing through a mixer. The mixer combines the output signals, which are next applied to a switch that selects an output modality according to a bipolar or quadripolar therapy. This apparatus applies a single mixed low-frequency signal to the pair of electrodes.

The document US 2010/0152817 A1 is known, which proposes a network simulator for the electrical stimulation of the nerves by pairs of electrodes successively or in alternation according to various application modes. The simulator produces electrical pulses of such a nature that the signals are transmitted sequentially to successive pairs of electrodes in a cycle so that the respective pairs of electrodes receive the corresponding signals at different moments, i.e. in alternation so that the signals are received by the pairs of electrodes so that they do not commence and end all at the same time. This simulator proposes, by means of varied applications of electrical pulses, to generate a longer or higher-amplitude simulation pulse. This simulator supplies one and the same signal to each pair of electrodes.

The document US 2003/0181960 A1 is also known, which proposes an electrotherapy apparatus which, from a first signal and a second signal, generates a therapeutic signal administered by an electrode and recovered by a return electrode. This apparatus does not make it possible to administer varied treatments. This apparatus operates in the low frequencies.

There is therefore a need to propose a solution that allows optimised treatments that are in particular less risky and more effective for the user who receives the treatment and simplified for the practitioner administers the treatment.

The other objects, features and advantages of the present invention will appear upon examining the following description and the accompanying drawings. It is understood that other advantages can be incorporated.

### SUMMARY

To achieve this objective, according to one embodiment, an electrotherapy device is provided comprising:
N electrodes, N being equal to 2 or 3,
two sinusoidal-voltage generators, advantageously intended to produce respectively a first signal and a second signal, characterised in that
the device comprises a transformer for each voltage generator so that the two voltage generators are isolated by a transformer, and
the device comprises a control unit configured so that the device takes in alternation:
   a first so-called series configuration wherein the N equal to 2 electrodes being active electrodes and the two voltage generators are put in series, the device does not comprise a return electrode, each active electrode being connected to a voltage generator, the device is configured to generate a different potential at each active electrode so as to provide the transmission of the current between the two closest active electrodes,
   a second so-called parallel configuration wherein N is equal to 3, two electrodes of which are active electrodes each respectively connected to a voltage generator and one electrode is a return electrode to form the ground and is connected to each voltage generator, the two voltage generators are put in parallel, the device is configured to generate a different potential at each active electrode so as to provide the transmission of the current between the two closest active electrodes and/or between each active electrode and the return electrode.

With this invention, it is therefore possible to apply a plurality of signals through a plurality of electrodes on the body of the user, this is a bi-channel device. The practitioner can implement a complete treatment offering diathermy and/or conductivity by parallelising or serialising the two voltage generators. The presence of two isolated generators ensures use without risk for the user, who is isolated on an electrical circuit. Moreover, the possibility of acting on the currents diverted between the active electrodes to provide the treatment of the user makes it possible to increase the surface areas of treated zones and thus to offer a multiple treatment. This choice is very surprising, since conventionally it is sought to reduce the currents diverted between active electrodes to focus on the currents between active electrodes and return or neutral electrode. Advantageously, each active electrode is connected to a voltage generator so as to receive respectively the first signal or the second signal produced by each voltage generator. Preferentially, the first signal and the second signal are different.

Another aspect relates to a method for operating a device as described above comprising:
a first operating mode according to the first configuration of the device in which N electrodes, N being equal to 2, are active, each connected to a voltage generator, the two voltage generators are connected in series, the current circulating between the two active electrodes,
a second operating mode according to the second configuration of the device in which, N being equal to 3, two electrodes of which are active, each connected to a voltage generator, one electrode is a return electrode, the two voltage generators are connected in parallel, the current circulating between the closest active electrodes and between the active electrodes and the return electrode.

### BRIEF DESCRIPTION OF THE FIGURES

The aims, objects, characteristics and advantages of the invention will emerge more clearly from the detailed description of one embodiment thereof, which is illustrated by means of the following accompanying drawings, in which:

FIG. 1 shows the electrical diagram of a device according to a first embodiment of the invention according to the first configuration with two active electrodes and two voltage generators.

FIG. 2 shows the circulation of the currents between the electrodes of a device according to FIG. 1 applied to a human body.

FIG. 3 shows the electrical diagram of a device according to a first embodiment of the invention according to the second configuration with two active electrodes, a neutral return electrode and two voltage generators.

FIG. 4 and FIG. 5 show the circulation of the currents between the electrodes of a device according to FIG. 3 applied to a human body. FIG. 4 illustrates the circulation of the currents between two active electrodes when the generators are synchronised. FIG. 5 illustrates the circulation of the currents between two active electrodes and between the active electrodes and the return electrode when the generators are desynchronised.

FIG. 6 shows the electrical diagram of a device according to a second embodiment of the invention according to the first configuration, with two active electrodes also being return electrode and two voltage generators.

FIG. 7 shows the circulation of the currents of a device according to FIG. 6 applied to the human body.

### DETAILED DESCRIPTION

Before giving a detailed review of embodiments of the invention, optional features are set out below, which can optionally be used as an alternative to or in combination with one another.

According to one example, each voltage generator is configured to generate a high-frequency voltage, preferentially between 100 kHz and 10 MHz.

According to one example, the active electrodes are configured to be movable.

According to one example, the active electrodes are capacitive or resistive or multi-pole.

According to one example, each voltage generator comprises a measuring member configured to measure output parameters of the voltage generator.

According to one example, each voltage generator comprises a control module configured to control the output current and/or the frequency and/or the dephasing of the voltage generator.

The control module is for example a microcontroller or a microprocessor or a programmable logic circuit (CPLD Complex Programmable Logic Device) or an in situ array of programmable gates (Field Programmable Gate Array FPGA) or an analogue circuit.

According to one example, the measuring member communicates with the control module to supply to it data necessary for the control. Advantageously, the voltage generator can control its output according to the impedance measured by the measuring member.

According to one example, the device according to the invention comprises a synchronisation member configured to control the synchronisation or the desynchronization of the voltage generators.

According to one example, the device does not comprise a switch arranged between an output of a voltage generator and an active electrode.

According to one example, the device does not comprise a mixing member arranged between an output of a voltage generator and an active electrode.

According to one example, the method according to the invention comprises a step of controlling the phasing or dephasing of the two generators advantageously by the control module, and comprising a step of controlling the synchronisation, by transmitting a synchronisation or desynchronisation signal, of the two generators advantageously by the synchronisation member so as to generate a current circulating between the two active electrodes.

The present invention relates to an electrotherapy device able to provide treatment by diathermy and/or conductivity of the body of the user.

Advantageously, the electrotherapy device according to the invention makes it possible to treat the body of the user by diathermy or conductivity or by diathermy combined with conductivity.

Advantageously, the various treatments are performed by putting in series, which is also referred to as serialisation, or putting in parallel, which is also referred to as parallelisation, the voltage generators of the device.

The electrotherapy device according to the invention comprises a plurality of electrodes, i.e. a number of electrodes equal to two or three. The number of electrodes of the device is preferentially defined by the number N, N being equal to 2 or 3, According to one possibility, the number of electrodes is equal to two or three or more generally to an even or odd number.

The electrotherapy device according to the invention also comprises two sinusoidal-voltage generators 210, 220.

Preferentially, the number of sinusoidal-voltage generators 210, 220 of the device is defined with respect to the number of electrodes and more particularly the active electrodes 212, 222.

According to one possibility in which the number of active electrodes is an even number, the number of sinusoidal-voltage generators is equal to this number of electrodes.

The device comprises two channels, each channel comes from a voltage generator 210, 220.

According to one possibility, each voltage generator 210, 220 comprises at least one channel referred to as a transmission channel corresponding to an output of the signal of the generator.

The electrodes are active electrodes 212, 222, i.e. the electrode supplies a current to the body of the user 10. Preferentially, the two active electrodes 212, 222 are connected to a different channel. Advantageously, each active electrode 212, 222 is connected to a voltage generator, more precisely to a transmission channel of a voltage generator. The device can also comprise, depending on the configuration, a return or neutral electrode 240, i.e. an electrode receiving the current that was transmitted by the active electrodes 212, 222 and passed through a portion of the body of the user 10. According to one possibility, each voltage generator 210, 220 comprises a so-called reception channel corresponding to an input receiving the current that passed through a portion of the body of the user 10. The return electrode 240 provides the closure of the electrical circuit at the body of the user 10. The return electrode 240 forms the ground. The return electrode 240 is preferentially fixed, but may be movable depending on the treatments. Fixed means that, during the treatment, the return electrode is not moved by the practitioner, the electrode can be kept fixed on the user by holding means. Movable means that, during the treatment, the electrode is moved by the practitioner.

The electrodes 212, 222, 240 are configured to be applied to the body of the user 10.

Advantageously, the electrodes 212, 222, 240 are in contact with the body of the user 10.

The active electrodes 212, 222 can be capacitive, resistive or multi-pole. Multi-pole means that the active electrode 212, 222 comprises two conductive poles, such as a capacitive pole and a resistive poll simultaneously.

The active electrodes 212, 222 can be fixed or movable depending on the requirements of the treatment.

The electrotherapy device according to the invention also comprises a control unit configured so that the device alternately takes a first configuration and a second configuration. The control unit is for example a microcontroller or a microprocessor or a programmable logic circuit (CPLD Complex Programmable Logic Device) or an in situ array of programmable gates (Field Programmable Gate Array FPGA) or an analogue circuit.

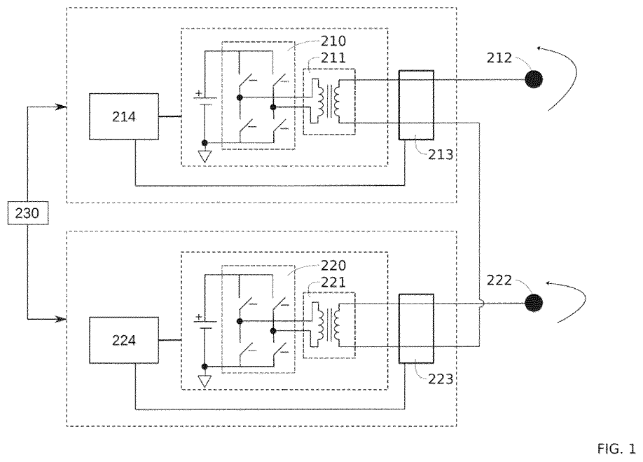

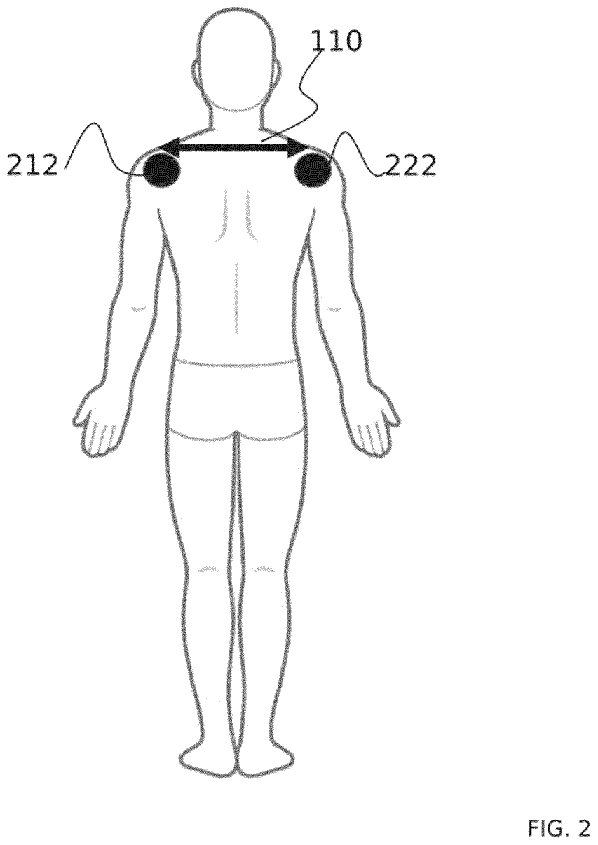

According to a first configuration, the device comprises N electrodes, with N equal to 2, which are active electrodes 212, 222, the device does not comprise a return electrode 240. Thus each electrode 212, 222 is connected to a voltage generator 210, 220. The two voltage generators 210, 220 are arranged in series. This configuration is illustrated in FIG. 1 and in FIG. 2.

According to a second configuration, the device comprises N electrodes, with N equal to 3, two electrodes of which are active electrodes 212, 222, and one electrode is a return electrode 240.

Thus, each active electrode 212, 222 is connected to a voltage generator 210, 220. The return electrode 240 is connected to the common point of the two voltage generators 210, 220.

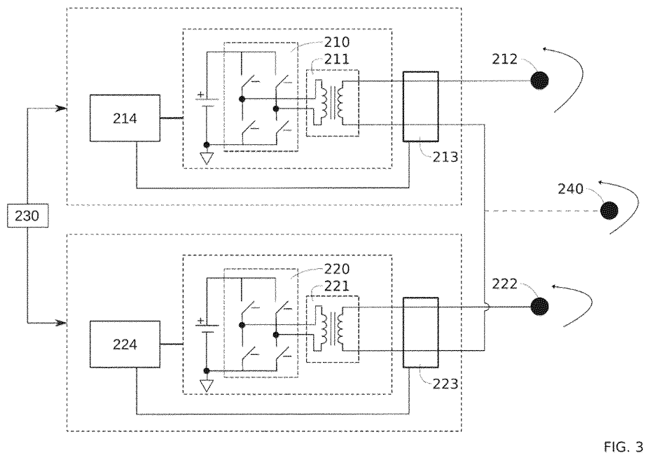

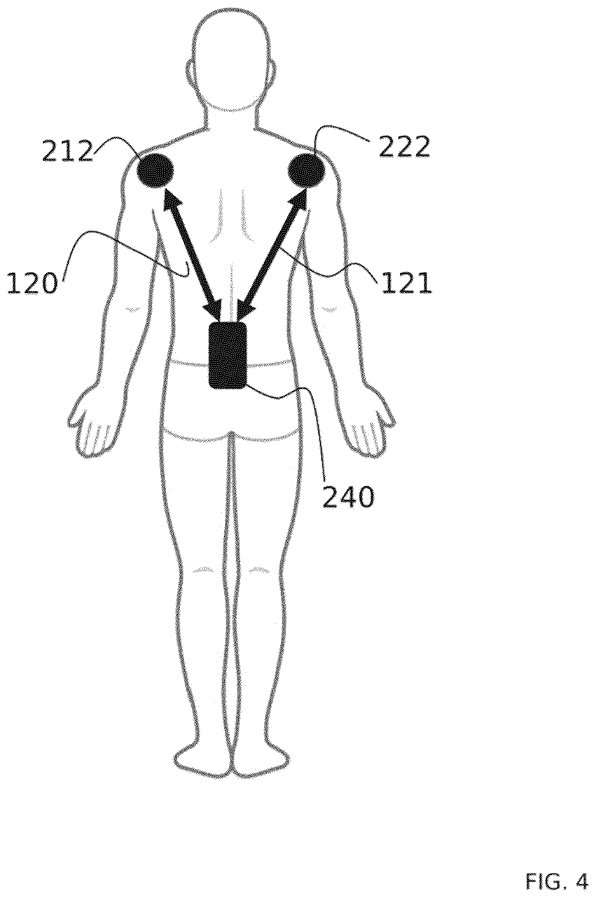

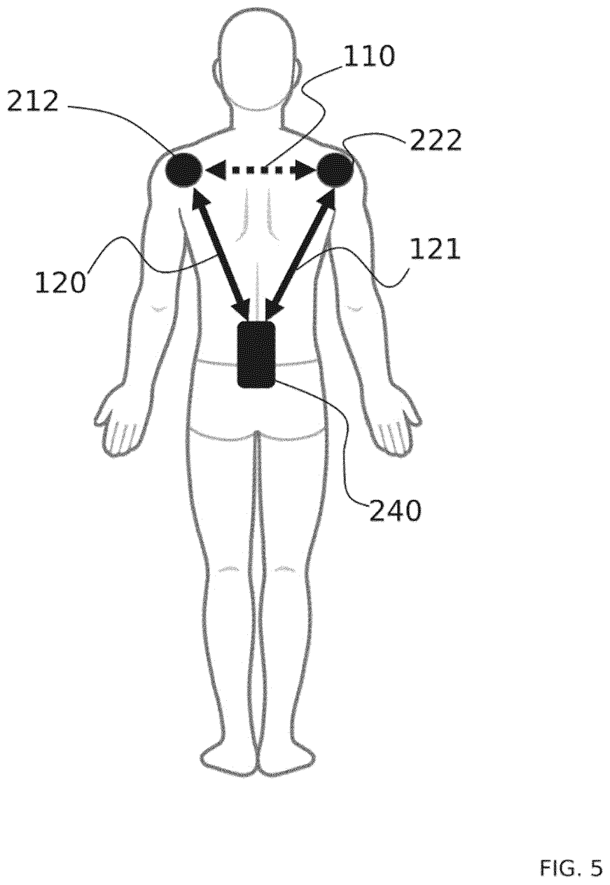

Preferentially, the common point of the two voltage generators 210, 220 is connected to each of the reception channels of each generator. The two voltage generators 210, 220 are arranged in parallel. This configuration is illustrated in FIG. 3 and in FIGS. 4 and 5.

According to a preferred embodiment, the voltage generators 210, 220 are isolated. The voltage generators 210, 220 are isolated galvanically. According to one embodiment shown on the figures, the isolation of the voltage generators 210, 220 is implemented by a transformer 211, 221 arranged at the output of each voltage generator 210, 220. The isolated voltage generator provides great safety for the user who receives a current while being isolated from the electrical circuit of the device.

The transformers 211, 222 are advantageously configured to be in phase or out of phase, in particular in opposition, depending on the requirement for the signals transmitted by the voltage generators 210, 220 to be in phase or out of phase.

According to the invention, the device thus makes it possible to use or not a return electrode 240.

The device according to the invention uses the diverted currents circulating between the active electrodes 212, 222, preferentially between the two active electrodes 212, 222. These diverted currents are conventionally reduced or even prevented by the devices of the prior art.

Advantageously, each active electrode 212, 222 is respectively connected to a sinusoidal-voltage generator 210, 220, preferentially by a respective channel. Preferentially, the device does not comprise a switch intended to pass a first signal of a voltage generator 210 to the two active electrodes or a second signal of a second voltage generator 210 to the two active electrodes.

Advantageously, the device does not comprise a mixing member intended to combine the first signal of the first generator and the second signal of the second generator.

Preferentially, the signal produced by each generator is applied to an active electrode.

The device according to the invention is configured to generate a different potential at each electrode 212, 222 so as to provide the transmission of the current between the two closest electrodes 212, 222, 240.

According to the invention, the neutral or floating common points at all the active electrodes 212, 222 is connected or not to a return electrode 240 applied to the body of the user 10 depending on the configuration.

The method using the device generates, according to the first configuration, a compound voltage 110 between the active electrodes 212, 222 and/or, according to the second configuration, simple voltages 120, 121 between the active electrodes 212, 222 and the return electrode 240 applied to the body of the user 10.

According to a preferred embodiment, the device is intended for electrotherapy and more precisely for diathermy. For this purpose, each voltage generator 210, 220 is configured to generate a high-frequency voltage. The high-frequency voltage is preferentially between 100 kHz and 10 MHz.

Advantageously, the device comprises, for each voltage generator 210, 220, a measuring member 213, 223. The measuring member 213, 223 is configured to measure the parameters of the signal output from the isolated voltage generator 210, 220.

Preferentially, the device comprises, for each voltage generator 210, 220, a control module 214, 224. The control module 214, 224 is configured to control the signal output from the isolated voltage generator 210, 220. Preferentially, the control module 214, 224 regulates for example the frequency of the current and/or the dephasing of the voltage generator. By way of example, the control module 214, 224 is a microcontroller or a microprocessor or a programmable logic circuit (CPLD Complex Programmable Logic Device) or an in situ array of programmable gates (Field Programmable Gate Array FPGA) or an analogue circuit.

Advantageously, the measuring member 213, 223 is configured to communicate with the control module 214, 224 so as to supply to it the data necessary for the control.

Preferentially, the device comprises a synchronisation member 230 advantageously arranged so as to control the voltage generators 210, 220. The synchronisation member 230 is arranged on a synchronisation input of each voltage generator 210, 220. The synchronisation member 230 is configured to allow the synchronisation or the desynchronization of the voltage generators 210, 220 and therefore of the signals transmitted. Synchronisation means that the signals transmitted have the same angular frequency and/or same frequency and/or same period. The synchronisation member 230 generates for example a synchronisation signal for each generator so as to control the synchronisation or desynchronisation of the signals. As illustrated in FIGS. 4 and 5, when the two voltage generators 210, 220 are synchronised, the currents circulate between each active electrode 212, 222 and the neutral electrode 240 while, when the two voltage generators 210, 220 are desynchronised, the currents also circulate between the two active electrodes 212, 220, it is the diverted current 110. The control of the synchronisation or desynchronisation of the signals participates in forming and maintaining the diverted current between the two active electrodes.

Advantageously, the voltage generators 210, 220 are independent and can in particular adjust the output signal according to the impedance seen by the device. The device according to the invention is advantageously configured to be a multi-output device, i.e. comprising two active electrodes connected to voltage generators generating distinct output signals.

Advantageously, the signal transmitted for each voltage generator 210, 220 is sinusoidal.

Preferentially, V=Vamp×sin (wt+Φ) with Vamp=Amplitude of the sinusoidal signal, w: angular frequency, Φ: phase difference in degrees.

According to a preferred embodiment, the device is configured to allow phasing or dephasing of the signals of each voltage generator 210, 220. Thus, the output voltage varies according to the phase difference of the waves. When the signals of the voltage generators 210, 220 are in phase, the voltage between two active electrodes 212, 222 is twice the output voltage Vₒᵤₜ. (V₁₁₀=2×Vₒᵤₜ) with Vout: output voltage of the voltage generator. When the signals of the voltage generators 210, 220 are out of phase by 180° then the output voltage is zero, Vₐₘₚ=0V with Vamp: Amplitude of the output sinusoidal signal.

According to the first embodiment in its first configuration, the device according to the invention is illustrated in FIG. 1.

According to this first configuration of this first embodiment, N is equal to 2, with two active electrodes 212, 222 each respectively connected to a voltage generator 210, 220. The device according to this first configuration does not include a return electrode. In this first configuration, the voltage generators 210, 220 are arranged in series.

The voltage generators 210, 220 are advantageously isolated by means of a transformer 211, 222 respectively arranged at the output of each voltage generator 210, 220.

The transformers 211, 222 are advantageously configured to be in phase or out of phase, in particular in opposition, depending on the requirement for the signals to be in phase or out of phase.

The device according to this first embodiment comprises two measuring members 213, 223 arranged at the output of each voltage generator 210, 220. Each measuring member 213, 223 is connected to the respective control module 214, 224 of each voltage generator 210, 220.

In this embodiment according to this first configuration, in the absence of the return electrode 240, the current circulating is the diverted current 110 between the two active electrodes 212, 222 applied to the body of a user 10, as illustrated in FIG. 3.

In this first configuration, the signal is said to be compound:
V₁₁₀=V₁₂₀+V₁₂₁. Wherein V₁₁₀ is the voltage of the current 110 circulating between the two active electrodes 212, 222, V₁₂₀ is the voltage of the current 120 circulating between an active electrode 212 and a neutral electrode 240 not present, V₁₂₁ is the voltage of the current 121 circulating between an active electrode 222 and a neutral electrode 240 not present.

In this first configuration, putting the two isolated voltage generators 210, 220 in series enables the voltage to increase up to twice its nominal value to seek the breakdown effect of the insulation, i.e. an increase in the permeability of the hard biological tissues (bones, ligaments, etc.) and for the current to circulate therein.

This first configuration makes it possible to offer treatment by serialised diathermy. This configuration makes it possible to put the two voltage generators 210, 220 in series and thus to apply signals generating diathermy. This treatment is implemented by two active electrodes and the return electrode is said to be floating since it is not applied to the user. This arrangement increases the voltage and not its intensity in order for the current to maximise the conductivity of the biological tissues and therefore the acceleration of healing, while minimising heating thereof.

The aim is to favour cell metabolism of said tissues with voltages that may range up to 800 Vrms.

In this first configuration, the voltage generators 210, 220 are configured so that the signals transmitted are out of phase and synchronous.

According to this first embodiment of the invention, the device can alternatively take a second configuration illustrated in FIG. 3.

According to this second configuration of this embodiment, N is equal to 3, with two active electrodes 212, 222 each respectively connected to a voltage generator 210, 220. The device according to this second configuration comprises a return electrode 240. The return electrode 240 is advantageously connected to the common point of the voltage generators 210, 220. In this second configuration, the voltage generators 210, 220 are arranged in parallel.

In this embodiment, according to this second configuration comprising a return electrode 240, the current circulates between the active electrodes 212, 222 and a return electrode 240, but also advantageously between the two active electrodes 212, 222 as illustrated in FIG. 4.

In this second configuration, the signal is said to be simple with the current 120, 121. Each voltage generator 210, 220 supplies a different impedance. The electrodes being applied to the body of the user 10, an impedance exists between the two active electrodes 212, 222. A voltage, V₁₁₀, is applied between the two active electrodes 212, 222. The voltage V₁₁₀ varies according to the proximity of the two active electrodes 212, 222 and according to the phase difference of the signals as indicated above.

In this second configuration, putting the two voltage generators 210, 220 in parallel enables the intensity to increase up to twice its nominal value to seek the heating of the tissues passed through, i.e. in other words a diathermy action. Bringing the active electrodes 212, 222 closer together or further away from each other varies the power.

This second configuration makes it possible to offer treatment by diathermy and conductivity combined. This configuration makes it possible to put the two voltage generators 210, 220 in parallel and thus to apply signals generating diathermy and conductivity. In this second configuration, the return electrode 240 is positioned on the body of the user 10 in order to create three current segments distributed between the three electrodes. The sum of the three current segments 110, 120, 121 corresponds to the power delivered by the electrotherapy device. In addition, this configuration increases the intensity and not the voltage, which favours heating (diathermy) of the biological tissues passed through with intensities that can range up to 4 amperes.

In FIGS. 4 and 5, the two active electrodes 212, 222 transmit a sinusoidal current the frequency and characteristics of which can be different. They converge towards a return electrode 240 via electrical segments where the current 120 circulates and where the current 121 circulates with the possibility of creating a third electrical segment where the current 110 circulates depending on the adjustments of the return electrode 240. The invention makes it possible to distribute the power emitted by a bi-phase device by means of three electrical segments of three electrodes 212, 222, 240 on the body of the user 10. Between each electrode 212, 222, 240 there is an electrical segment and the practitioner can define how to distribute the power between these three electrodes 212, 222, 240. The device is configured to synchronise or desynchronise via the dephasing of the signals.

In FIG. 4, the signals are synchronised and can be in phase or out of phase. There is no diverted current.

In FIG. 5, the signals are desynchronised and can be in phase or out of phase. There is a diverted current.

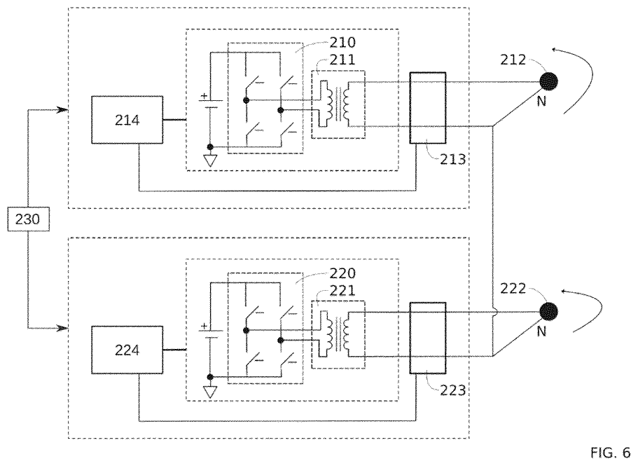

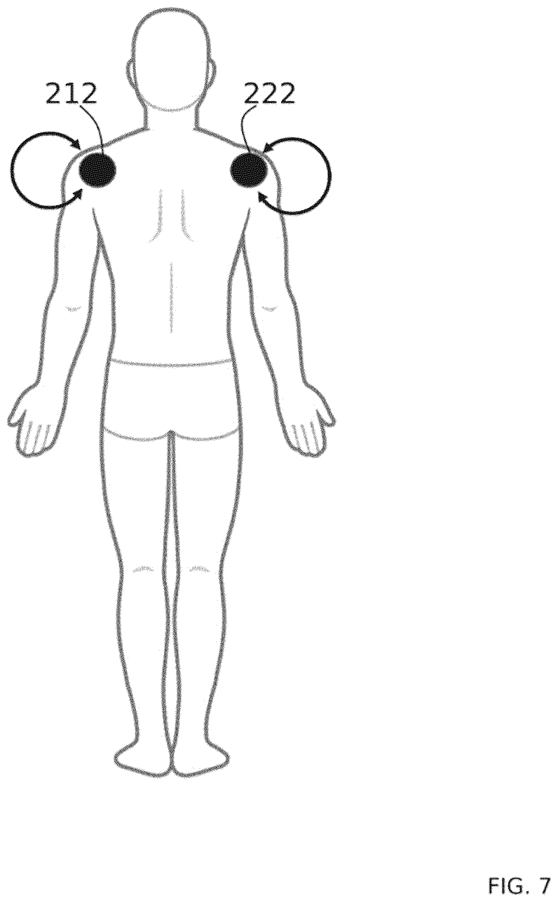

According to a second embodiment illustrated in FIG. 6, N is equal to 2, with two active electrodes 212, 222 each respectively connected to a voltage generator 210, 220. The device according to this second configuration does not include a return electrode. In this first configuration, the voltage generators 210, 220 are arranged in series. The active electrodes 212, 222 are advantageously configured to be simultaneously or in alternation active and neutral as illustrated in FIG. 6 and in FIG. 7 to provide circulation of the current input and output on a single electrode 212, 222.

According to one embodiment, the active electrodes may be smart electrodes. They are equipped with an inertial unit that makes it possible to know the movement of the practitioner in real time.

The practitioner can modify their treatment during use for better efficacy. It will also recognise the type of electrode used. The control module 214, 224 adapts the output according to the type of electrode detected.

By way of example, the device is advantageously designed for a maximum simple voltage of 400 Vrms, a maximum simple current of 1.2 Arms and a power of 150 W on frequencies ranging from 300 kHz to 1 MHz and a maximum compound voltage of 800 Vrms, a maximum simple current of 2.4 Arms and a power of 300 W on frequencies ranging from 300 kHz to 1 MHz.

The invention therefore advantageously makes it possible to vary the voltage and intensity of high-frequency currents and to work on different intensities and/or voltages and/or frequencies and to combine or not various types of electrodes.

According to one possibility, the voltage generators 210, 220 can operate to produce multifrequency voltages by modulating low-frequency, medium-frequency and high-frequency signals as described in an invention of the applicant. The voltage generator at the highest frequency is amplitude modulated by the voltage generator at the lowest frequency. The powers are unbalanced. Each generator must supply a power corresponding to its electrodes and to that of the associated electrodes. The power is adjusted according to the amplitude and phase difference of each generator.

According to this possibility, the electrotherapy device comprises a first voltage generator configured to generate a first voltage having a first frequency and comprising a first connection terminal and a second connection terminal, and a second voltage generator configured to generate a second voltage having a second frequency strictly higher than the first frequency and comprising a third connection terminal and a fourth connection terminal, characterised in that it comprises a transmission channel and a reception channel and in that the first connection terminal and the third connection terminal are connected to said transmission channel and in that the second connection terminal and the fourth connection terminal are connected to said reception channel.

This possibility makes it possible to apply two voltages of two different frequencies by one and the same transmission channel and one and the same reception channel.

More particularly, the electrotherapy device comprises a control unit configured to control the first voltage generator to generate the first voltage at a first frequency, and to control the second voltage generator the second voltage at a second frequency simultaneously.

Advantageously, the control unit is configured to generate in the transmission channel a signal associating a sinusoidal voltage of a first frequency modulated by a third frequency and associated with a sinusoidal voltage of a second frequency modulated by a fourth frequency, the signal being the sum of the voltage of the first generating module and of the voltage of the second generating module.

Another aspect relates to a method for operating an electrotherapy device as described for this possibility comprising:
a. generating a first voltage at a first frequency by the first generator, and
b. generating a second voltage at a second frequency strictly higher than the first frequency by the second generator, steps a and b being simultaneous. Thus the two voltage generators operate simultaneously.

According to one example, the first voltage generator is configured to generate a sinusoidal voltage.

According to one example, the first frequency lies in a first frequency interval between 1 kHz and 10 kHz.

According to one example, the first voltage generator is configured to generate a sinusoidal voltage of a first frequency modulated by a third frequency.

According to one example, the third frequency lies in a third frequency interval between 1 Hz and 150 Hz.

According to one example, the third frequency is a sinusoidal voltage.

This possibility of the device proposes to create a medium-frequency sinusoidal signal in order to modulate the low-frequency signal (electrostimulation) and to combine it with a high-frequency sinusoidal signal (diathermy). The device according to this possibility makes it possible to combine, within a single channel, the advantages of electrostimulation and of radiofrequency generating diathermy. The device according to this possibility produces a non-invasive current stimulating the natural healing mechanisms of the body and favouring cell exchange. It offers excellent rehabilitation results by virtue of rapid recovery of the muscle and joint functions.

The result is a sinusoidal current of 1 kHz to 10 kHz modulated at a frequency of 1 Hz to 150 Hz.

In electrostimulation, modulation of the stimulating current makes it possible to avoid tetany of the excited muscles.

According to one example, the second voltage generator is configured to generate a sinusoidal voltage.

According to one example, the second frequency lies in a second frequency interval between 100 kHz and 10 MHz.

According to one example, the second voltage generator is configured to generate a sinusoidal voltage of a second frequency modulated by a fourth frequency According to one example, the fourth frequency lies in a fourth frequency interval between 1 Hz and 150 Hz.

According to one example, the fourth frequency is of the pulse type.

According to one example, the second voltage generator comprises an activation command configured to generate a pulse sinusoidal voltage at the fourth frequency.

According to one example, the first voltage generator comprises a transmission command configured to transmit the voltage of the first generating module at the fourth frequency.

The invention is not limited to the aforementioned embodiments, and includes all the embodiments covered by the application.

### LIST OF REFERENCES

10. Body of the user
110. Current diverted between two active electrodes
111. Current diverted between two active electrodes
112. Current diverted between two active electrodes
120. Current diverted between an active electrode and a neutral electrode
121. Current diverted between an active electrode and a neutral electrode
122. Current diverted between an active electrode and a neutral electrode
210. Voltage generator
211. Transformer
212. Active electrode connected to the first voltage generator
213. Measuring member
214. Control module
220. Voltage generator
221. Transformer
222. Active electrode connected to the second voltage generator
223. Measuring member
224. Control module
230. Synchronisation member
240. Neutral or return electrode

1. An electrotherapy device comprising:
N electrodes, N being equal to 2 or 3,
two sinusoidal-voltage generators,
wherein
the device comprises a transformer for each voltage generator so that the two voltage generators are isolated by a transformer, and
the device comprises a control unit configured so that the device takes in alternation:
a first so-called series configuration wherein the N equal to electrode being active electrodes and the two voltage generators are put in series, the device does not comprise a return electrode, each active electrode being connected to a voltage generator, the device is configured to generate a different potential at each active electrode so as to provide the transmission of the current between the two closest active electrodes,
a second so-called parallel configuration wherein N is equal to 3, two electrodes of which are active electrodes each respectively connected to a voltage generator and one electrode is a return electrode to form the ground and is connected to each voltage generator, the two voltage generators are put in parallel, the device is configured to generate a different potential at each active electrode so as to provide the transmission of the current between the two closest active electrodes and/or between each active electrode and the return electrode.

2. The device according to claim 1, wherein each voltage generator is configured to generate a high-frequency voltage.

3. The device according to claim 1, wherein each active electrode is connected to a voltage generator so as to receive respectively a first signal or a second signal produced respectively by a voltage generator.

4. The device according to any one of the preceding claim 1, wherein the active electrodes are configured to be movable.

5. The device according to any one of the preceding claim 1, wherein the active electrodes are capacitive or resistive or multi-pole.

6. The device according to any one of the preceding claim 1, wherein each voltage generator comprises a measuring member configured to measure output parameters of the voltage generator.

7. The device according to any one of the preceding claim 1, wherein each voltage generator comprises a control module configured to control the output current and/or the frequency and/or the dephasing of the voltage generator.

8. The device according to claim 7, wherein the measuring member communicates with the control module to supply to it data necessary for the control.

9. The device according to claim 1 comprising a synchronisation member configured to control the synchronisation or the desynchronization of the voltage generators.

10. The device according to claim 9, wherein, in the first configuration, the signals transmitted by the voltage generators are out of phase and synchronous.

11. The device according to claim 1, wherein:
the first voltage generator is configured to generate a first voltage having a first frequency and the first voltage generator comprising a first connection terminal and a second connection terminal,
the second voltage generator is configured to generate a second voltage having a second frequency strictly higher than the first frequency and the second voltage generator comprising a third connection terminal and a fourth connection terminal,
a transmission channel,
a reception channel,
and the first connection terminal and the third connection terminal are connected to said transmission channel and the second connection terminal and the fourth connection terminal are connected to said reception channel,
a control unit configured to simultaneously:
control the first voltage-generating module to generate the first voltage at a first frequency, and
control the second voltage-generating module the second voltage at a second frequency,
the control unit being configured to generate in the transmission channel a signal associating a sinusoidal voltage at the first frequency modulated by the third frequency and associated with a sinusoidal voltage at the second frequency modulated by the fourth frequency, the signal being the sum of the voltage of the first generator and of the voltage of the second generator.

12. The device according to claim 3 wherein the first signal differs from the second signal.

13. The device according to claim 2, wherein each voltage generator is configured to generate a high-frequency voltage between 100 kHz and 10 MHz.

14. A process for electrotherapy by using the electrotherapy device according to claim 1 comprising the followings steps:
the control unit controls the device to take in alternation:
a first so-called series configuration wherein the N equal to 2 electrodes being active electrodes and the two voltage generators are put in series, the device does not comprise a return electrode, each active electrode being connected to a voltage generator, the device generates a different potential at each active electrode providing the transmission of the current between the two closest active electrodes,
a second so-called parallel configuration wherein N is equal to 3, two electrodes of which are active electrodes each respectively connected to a voltage generator and one electrode is a return electrode to form the ground and is connected to each voltage generator, the two voltage generators are put in parallel, the device generates a different potential at each active electrode providing the transmission of the current between the two closest active electrodes and/or between each active electrode and the return electrode.

15. A process for electrotherapy by using the electrotherapy device according to claim 11 comprising the followings steps:
The first voltage generator generates a first voltage having a first frequency and the first voltage generator comprising a first connection terminal and a second connection terminal,
the second voltage generator generates a second voltage having a second frequency strictly higher than the first frequency and the second voltage generator comprising a third connection terminal and a fourth connection terminal,
and the first connection terminal and the third connection terminal are connected to said transmission channel and the second connection terminal and the fourth connection terminal are connected to said reception channel,
the control unit simultaneously:
controls the first voltage-generating module to generate the first voltage at a first frequency, and
controls the second voltage-generating module the second voltage at a second frequency, and
the control unit generates in the transmission channel a signal associating a sinusoidal voltage at the first frequency modulated by the third frequency and associated with a sinusoidal voltage at the second frequency modulated by the fourth frequency, the signal being the sum of the voltage of the first generator and of the voltage of the second generator.
